(19) United States
(12) Patent Application Publication
(10) Pub. No.: US 2025/0375190 A1
(43) Pub. Date: Dec. 11, 2025

(54) NONINVASIVE MID-STREAM URINE COLLECTION PRODUCT FOR COGNITIVELY IMPAIRED AND/OR INCONTINENT INDIVIDUALS

(71) Applicant: Urovative Technologies LLC ( Wyoming registered LLC ), South San Francisco, CA (US)

(72) Inventors: Ara Sayabalian, Tarzana, CA (US); Hassan Zaidi, South San Francisco, CA (US)

(21) Appl. No.: 19/221,232

(22) Filed: May 28, 2025

Related U.S. Application Data
(60) Provisional application No. 63/656,400, filed on Jun. 5, 2024.

(51) Int. Cl. A61B 10/00; A61F 13/505; A61F 13/53; A61F 13/537; A61L 15/26; A61L 15/40; A61L 15/60

(52) U.S. Cl. CPC A61B 10/007; A61F 13/505; A61F 13/53708; A61L 15/26; A61L 15/40; A61L 15/60; A61F 2013/530029; A61F 2013/530481

(57) ABSTRACT

An absorbent article may include a wick material positioned to receive a liquid insult, wherein the liquid insult comprises a first volume and a second volume. The absorbent article may include one or more liquid collection units positioned underneath the wick material, wherein the wick material distributes the first volume away from the one or more liquid collection units, the one or more liquid collection units comprising: an initial absorbent material configured to receive the second volume flowing through the wick material, a sample absorbent material configured to: receive the second volume flowing through the initial absorbent material; and store the second volume; and a liquid-restrictive layer. The absorbent article may include a liquid absorbent core, wherein the one or more liquid collection units are disposed on or in a front side of the liquid absorbent core.

### CROSS-REFERENCE TO RELATED APPLICATIONS

The present application claims the benefit under 35 U.S.C. § 119(e) of U.S. Provisional Patent Application Ser. No. 63/656,400, filed Jun. 5, 2024, which is incorporated herein by reference in its entirety.

### TECHNICAL FIELD

The present invention generally relates to urine collection devices for testing, and, in particular, to a wearable noninvasive urine collection device.

### BACKGROUND

Urine analysis is crucial in diagnosing and monitoring various medical conditions, from urinary tract infections (UTIs) to kidney disease. Urine analysis is also useful when analyzing diabetes, sediment concentrations, ketones, and other urine constituents. However, collecting uncontaminated urine samples, also referred to as specimen, from individuals who are cognitively impaired and/or frequent-to-fully incontinent presents unique challenges, especially as it relates to the diagnosis of urinary tract infections (UTIs). Traditional urine collection methods, such as clean catch or midstream catches, are often impractical for these populations due to difficulties in communication, mobility, or understanding of the process required to obtain a clean sample. The gold standard for a clean catch is through catheterization. However, catheterization is difficult, time-consuming, invasive, and expensive. Additionally, catheterization often needs to be done with two or more individuals.

Currently, adult disposable absorbent product swabs are commonly used as a method to collect urine from adults who are either incontinent and/or have cognitive impairments. Collecting a urine sample via swab includes pushing the swab against a used, or insulted, absorbent product, which saturates the swab, and then placing the urine-saturated swab inside a vial for delivery. The swab method is often favored by individuals in charge of collecting the samples because it is difficult to have a cognitively impaired individual or incontinent individual urinate into a sterile cup. However, the swab method has significant limitations. Swabs are highly susceptible to contamination from fecal matter, skin flora, or other substances in the disposable absorbent product. This contamination can lead to misleading laboratory results, including false positives for infection and the presence of additional bacteria and other microbes such as fungi (e.g., yeasts) not found in the urine. The contaminated samples can result in incorrect diagnoses, which can lead to inappropriate antibiotic prescribing. Inappropriate antibiotic prescribing can contribute to antibiotic resistance, a growing global health concern.

Therefore, it is desirable to provide a system and method that overcomes the shortfalls of the previous approaches discussed above.

### SUMMARY

An absorbent article is disclosed. In one or more embodiments, the absorbent article includes a wick material positioned to receive a liquid insult, wherein the liquid insult includes a first volume and a second volume, and one or more liquid collection units positioned underneath the wick material, wherein the wick material distributes the first volume away from the one or more liquid collection units. In one or more embodiments, the one or more liquid collection units includes an initial absorbent material configured to receive the second volume flowing through the wick material, and a sample absorbent material configured to receive the second volume flowing through the initial absorbent material and store the second volume. In one or more embodiments, the absorbent article incudes a liquid-restrictive layer and a liquid absorbent core, wherein the one or more liquid collection units are disposed on or in a front side of the liquid absorbent core.

In one or more embodiments of the absorbent article, the wick material further covers a back side of the initial absorbent material.

In one or more embodiments, the absorbent article further includes an adhesive layer applied on an exterior side.

In one or more embodiments of the absorbent article, the liquid absorbent core includes an absorbent agent.

In one or more embodiments of the absorbent article, the absorbent agent includes superabsorbent polymer (SAP).

In one or more embodiments of the absorbent article, the sample absorbent material includes an absorbent textile.

In one or more embodiments of the absorbent article, the absorbent textile includes at least one of cotton, rayon, and polyester.

In one or more embodiments of the absorbent article, the absorbent textile includes more than 90% cotton.

In one or more embodiments of the absorbent article, the sample absorbent material is configured to be removed from the one or more liquid collection units after the liquid insult and inserted into a syringe barrel.

In one or more embodiments of the absorbent article, when the sample absorbent material is removed after a saturating liquid insult a recovery volume equal to or more than two milliliters of liquid can be removed from the sample absorbent material.

In one or more embodiments of the absorbent article, the first volume is equal to or more than five milliliters.

In one or more embodiments of the absorbent article, when the sample absorbent material is removed after a saturating liquid insult a recovery volume equal to or more than two milliliters of the second volume can be removed from the sample absorbent material, wherein the first volume is equal to or more than five milliliters.

In one or more embodiments of the absorbent article, the initial absorbent material includes a preservative.

In one or more embodiments of the absorbent article, the preservative includes at least one of boric acid, monalazone disodium, and sodium bicarbonate.

In one or more embodiments of the absorbent article, the initial absorbent material includes at least 90% cotton.

In one or more embodiments of the absorbent article, the liquid-restrictive layer includes a nonpermeable layer.

In one or more embodiments of the absorbent article, the liquid-restrictive layer includes polyurethane.

In one or more embodiments of the absorbent article, the wick material includes nonwoven material.

In one or more embodiments of the absorbent article, the absorbent article is configured as a wearable garment.

A method is disclosed. In one or more embodiments, the method includes receiving a swab containing a mid-stream urine specimen from a liquid collection unit of an absorbent article. In one or more embodiments, the method includes placing the swab in a syringe barrel. In one or more embodiments, the method includes applying a plunger to the syringe barrel. In one or more embodiments, the method includes collecting the mid-stream urine specimen.

It is to be understood that both the foregoing general description and the following detailed description are exemplary and explanatory only and are not necessarily restrictive of the present disclosure. The accompanying drawings, which are incorporated in and constitute a part of the specification, illustrate subject matter of the disclosure. Together, the descriptions and the drawings serve to explain the principles of the disclosure.

### BRIEF DESCRIPTION OF THE DRAWINGS

The detailed description is described with reference to the accompanying figures. The use of the same reference numbers in different instances in the description and the figures may indicate similar or identical items. Various embodiments or examples (“examples”) of the present disclosure are disclosed in the following detailed description and the accompanying drawings. The drawings are not necessarily to scale. In general, operations of disclosed processes may be performed in an arbitrary order, unless otherwise provided in the claims.

FIG. 1A illustrates a perspective view of an absorbent article packed within a sanitary wrap, in accordance with one or more embodiments of the disclosure.

FIG. 1B illustrates a view of an interior side of the absorbent article, in accordance with one or more embodiments of the disclosure.

FIG. 1C illustrates a view of an exterior side of the absorbent article, in accordance with one or more embodiments of the disclosure.

FIG. 1D illustrates a perspective side view of the absorbent article with the interior side facing upwards and a side border cut away, exposing layers within the absorbent article, in accordance with one or more embodiments of the disclosure.

FIG. 2A illustrates the absorbent article with one of the one or more liquid collection units excised and placed adjacent to the absorbent article, in accordance with one or more embodiments of the disclosure.

FIGS. 2B and 2C illustrate obverse and reverse perspective views of a partially disassembled liquid collection unit, respectively, in accordance with one or more embodiments of the disclosure.

FIG. 2D illustrates a disassembled liquid collection unit, in accordance with one or more embodiments of the disclosure.

FIG. 3 illustrates a process flow diagram depicting a method for acquiring a urine specimen, in accordance with one or more embodiments of the disclosure.

FIG. 4 illustrates a process flow diagram depicting a method 400 for recovering the second volume from the absorbent article, in accordance with one or more embodiments of the disclosure.

### DETAILED DESCRIPTION

The present disclosure has been particularly shown and described with respect to certain embodiments and specific features thereof. The embodiments set forth herein are taken to be illustrative rather than limiting. It should be readily apparent to those of ordinary skill in the art that various changes and modifications in form and detail may be made without departing from the spirit and scope of the disclosure.

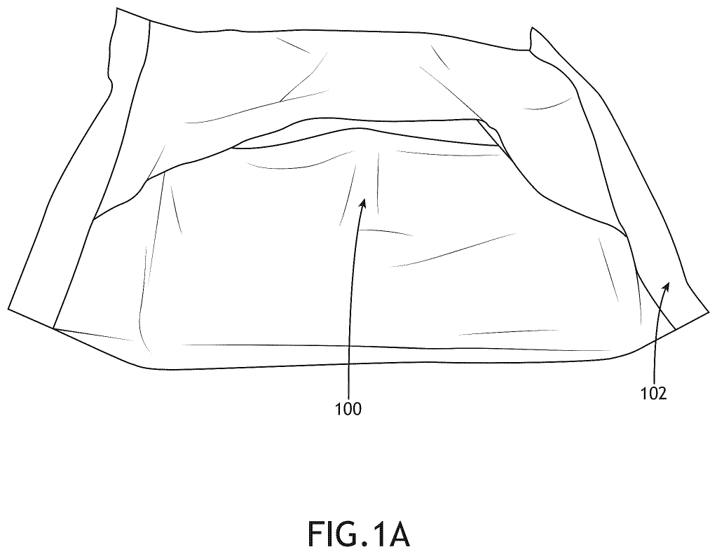

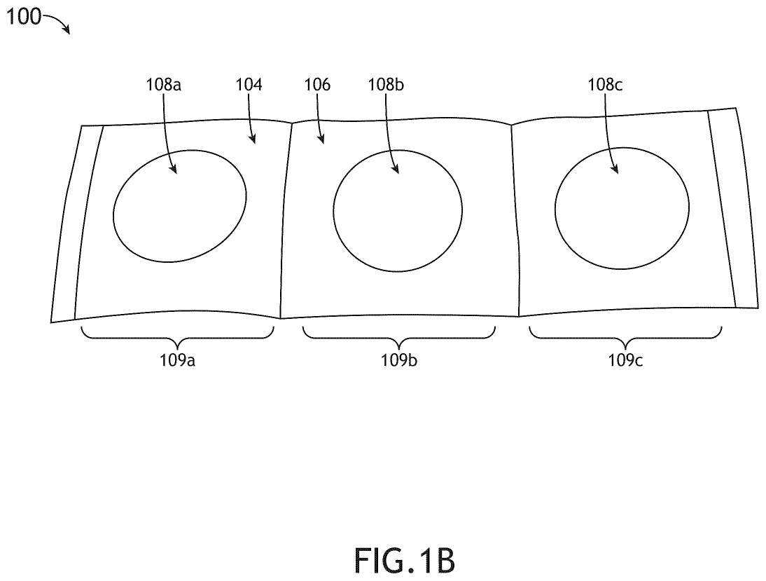

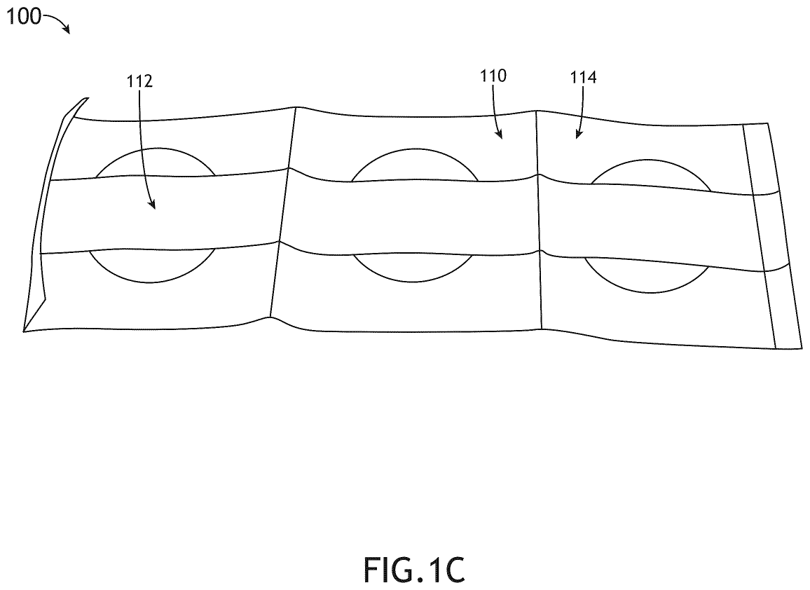

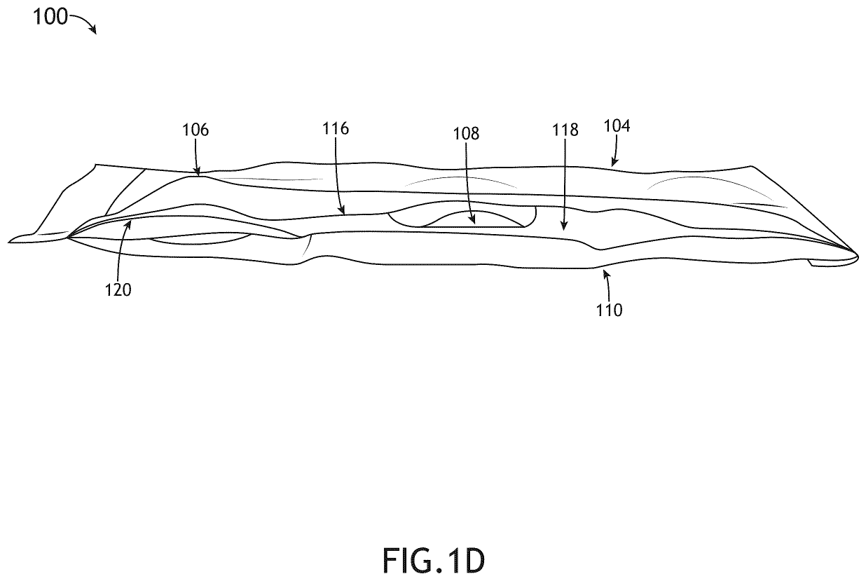

FIGS. 1A-3 generally illustrate an absorbent article for collecting a mid-stream urine specimen of an incontinent subject, in accordance with one or more embodiments of the disclosure.

Embodiments of the present disclosure are directed to an absorbent article that includes a liquid absorbent core, a wick material that distributes a first volume, or first catch, of urine (e.g., liquid) to the liquid absorbent core, and a liquid collection unit that receives a second volume, or mid-stream urine specimen, after the first volume has been distributed. The liquid collection unit includes a liquid-restrictive or impermeable layer disposed on the liquid absorbent core, a sample absorbent material disposed on the liquid-restrictive layer, and an initial absorbent material disposed on the sample absorbent material. The sample absorbent material absorbs the second volume of the urine specimen that is to be collected and tested. For testing, the liquid collection unit is removed from the absorbent article, the sample absorbent material is isolated from the liquid collection unit, and the urine specimen is then removed from the sample absorbent material.

Embodiments of the present disclosure are particularly advantageous for the collection of mid-stream urine specimens from moderate to severe cognitively impaired and/or frequent to fully incontinent individuals, as the mid-stream urine specimen is collected and isolated within the absorbent article without outside intervention or contamination. The midstream urine which is collected in the device is then easily transported to a laboratory facility. The entire device is shipped to the laboratory. The midstream urine specimen is then easily recovered from the absorbent article after removal of the absorbent article from the subject. The midstream urine is then recovered in a clean sterile environment. Once recovered, the midstream urine specimen has reduced contamination of both first-catch urine and non-urinary tract microbes as compared to other methods, resulting in an improved diagnosis and treatment plan.

FIG. 1A illustrates a perspective view of an absorbent article 100 packed within a sanitary wrap 102, in accordance with one or more embodiments of the disclosure. In embodiments, the absorbent article 100 is configured as a pad, such as a booster pad. In embodiments, the incontinence product is configured as a disposable incontinence garment, such as a diaper, disposable pull-on, or brief. In some embodiments, the absorbent article 100 is packed within a double sanitary wrap 102.

FIG. 1B illustrates a view of an interior side 104 (e.g., skin-facing side) of the absorbent article 100, in accordance with one or more embodiments of the disclosure. The interior side 104 of the absorbent article 100 also faces the urethra of the subject. In embodiments, the absorbent article includes a wick material 106. The wick material 106 receives urine (e.g., an insult) directly from the urethra of the subject and distributes urine along the interior side 104 of the absorbent article 100 for absorption by components of the absorbent article 100. For example, the wick material 106 may receive and distribute a first volume, or first catch, of urine, then receive and distribute a second volume, or mid-stream urine volume, of urine. The wick material 106 is also permeable, providing a material barrier between the subject and the urine as it is distributed to other components of the absorbent article. The wick material 106 may be of woven or unwoven material. For example, the wick material 106 may include one or more nonwoven layers that includes one or more of cellulose, cotton, rayon, and polyester.

The first volume of urine may be within a range of volumes. For example, the first volume may include a range of 2 milliliters (ml) to 40 ml of urine or an approximate range thereof, a range of 4 ml to 30 ml of urine or an approximate range thereof, or a range of 5 ml to 20 ml of urine or an approximate range thereof. For instance, the first volume of urine may be equal to or approximately 2 ml, may be equal to or approximately 4 ml, may be equal to or approximately 5 ml, may be equal to or approximately 10 ml, or may be equal to or approximately 20 ml.

In embodiments, the absorbent article 100 includes one or more liquid collection units 108a-c. The liquid collection units 108a-c collect midstream urine that includes a second volume of urine after the first volume of urine has been distributed. The absorbent article 100 may include any number or distribution of liquid collection units 108a-c. For example, the absorbent article 100 may include a single liquid collection unit 108. In another example, the absorbent article 100 may include two liquid collection units 108. In another example, the absorbent article 100 may include three liquid collection units 108 (e.g., as shown in FIG. 1B). In another example, the absorbent article 100 may include four or more liquid collection units 108. The liquid collection units 108a-b may be positioned within respective catch areas 109a-c.

In embodiments, the absorbent article 100 may include liquid collection units 108 of any size or shape. For example, the absorbent article 100 may include liquid collection units 108 having a circular, ovoid, rectangular, square, or other polygonal shape. In another example, the absorbent article 100 may include liquid collection units 108 having a diameter of approximately 4 cm, approximately 6 cm, or approximately 8 cm.

In embodiments, the absorbent article 100 may include catch areas 109a-c of any size or shape. For example, the absorbent article 100 may include catch areas 109a-c having a circular, ovoid, rectangular, square, or other polygonal shape. The catch areas 109a-c may be partially or entirely wrapped. For example, the catch areas 109a-c may be partially or entirely wrapped with one or more layers of permeable nonwoven material, such as the nonwoven material of the wick material 106.

The liquid collection units 108 may be configured to collect a range of second volumes of urine. For example, the collected second volume may include a range of 1 ml to 30 ml or an approximate range thereof. In another example, the second volume may include a range of 2 ml to 20 ml or an approximate range thereof. For instance, the collected volume of urine may be equal to or approximately 1 ml. In another instance, the second of urine may be equal to or approximately 2 ml. In another instance, the second volume of urine may be equal to or approximately 4 ml. In another instance, the second volume of urine may be equal to or approximately 10 ml. In another instance, the second volume of urine may be equal to or approximately 10 ml.

FIG. 1C illustrates a view of an exterior side 110 of the absorbent article 100, in accordance with one or more embodiments of the disclosure. In embodiments, the absorbent article 100 includes an adhesive layer or strip applied to the exterior side 110 and an adhesive strip cover 112. For example, when the adhesive strip cover 112 is removed, the adhesive strip is active, and the absorbent article 100 can be adhered to a disposable undergarment (e.g., underwear or absorbent garment) of a subject. The adhesive strip and adhesive strip cover 112 may be omitted if there is no need to adhere the absorbent article 100 to an undergarment, such as if the absorbent article 100 was configured as a wearable garment such as a diaper, disposable pull-on, or brief.

The exterior side 110 of the absorbent article 100 may include a backing layer 114. The backing layer 114 may include permeable material, semipermeable material, or nonpermeable material. For example, the backing layer 114 may include the same or similar material as used for the wick material 106. For instance, the backing layer 114 may include the same material as the wick material 106, with the material from the wick material 106 wrapped around both the interior side 104 and the exterior side 110 of the absorbent article 100. In another instance, one or more additional layers of wick material (e.g., unwoven material) may be partially or totally wrapped around the entire absorbent article 100.

FIG. 1D is a perspective side view of the absorbent article 100 with the interior side 104 facing upwards and a side border cut away, exposing layers within the absorbent article 100, in accordance with one or more embodiments of the disclosure. For example, the cut has separated the wick material 106 from the backing layer 114.

In embodiments, the absorbent article 100 includes a liquid absorbent core 116 disposed between the wick material 106 and the backing layer 114. The liquid absorbent core 116 absorbs the majority of the urine that can be absorbed by the absorbent article 100. The one or more liquid collection units 108 are disposed between the wick material 106 and the liquid absorbent core 116, with the one or more liquid collection units 108 disposed on or adjacent to a front side 118 of the liquid absorbent core 116, and opposite to a back side 120 of the liquid absorbent core 116. In another example, one or more liquid collection units may be distributed within the liquid absorbent core 116.

In embodiments, the liquid absorbent core 116 includes an absorbent agent associated with or integrated within a matrix. The absorbent agent may include superabsorbent polymer (SAP) absorbent agents including, but not limited to, polyacrylates, polyacrylamides, cellulose-based polymers, starch-acrylonitrile polymers, and maleic anhydride polymers. The matrix may include any material including but not limited to natural fibers (e.g., cotton or wood pulp) or artificial fibers (e.g., polyester or rayon).

In embodiments, the amount of absorbent agent or SAP included in the absorbent article 100 includes an amount that will not prevent urine from being collected by the liquid collection unit 108 while still preventing excess urine exposure to skin, causing a rash. For example, the liquid absorbent core 116 may include a range of SAP, such as a range of zero grams of SAP per absorbent article 100 to 20 grams of SAP per absorbent article 100, or an approximate range thereof. In another example, the liquid absorbent core 116 includes a range of 1 gram of SAP per absorbent article 100 to 10 grams of SAP per absorbent article 100, or an approximate range thereof. In another example, the liquid absorbent core 116 includes a range of 2 grams of SAP per absorbent article 100 to 5 grams of SAP per absorbent article 100 or an approximate range thereof. In another example, the liquid absorbent core 116 includes a range of 2 grams of SAP per absorbent article 100 to 4 grams of SAP per absorbent article 100, or an approximate range thereof. For instance, the amount of SAP per absorbent article may be equal to or approximately zero grams. In another instance, the amount of SAP per absorbent article may be equal to or approximately one gram. In another instance, the amount of SAP per absorbent article may be equal to or approximately two grams. In another instance, the amount of SAP per absorbent article may be equal to or approximately three grams. In another instance, the amount of SAP per absorbent article may be equal to or approximately four grams. In another instance, the amount of SAP per absorbent article may be equal to or approximately five grams. In another instance, the amount of SAP per absorbent article may be equal to or approximately six grams.

In embodiments, the wick material 106 distributes the first volume of urine away from the one or more liquid collection units 108 to the liquid absorbent core 116. For example, if an area of the wick material 106 directly above one or more liquid collection units 108 is insulted directly by urine, the wick material 106 will distribute the first volume of urine along the wick material 106, which is eventually absorbed by the liquid absorbent core 116. After the first volume has been distributed, a second volume of urine then insults the wick material 106. Because the wick material is saturated, the urine is no longer wicked away from the liquid collection unit 108, which then absorbs the second volume of urine, which is a portion of the midstream urine from the subject.

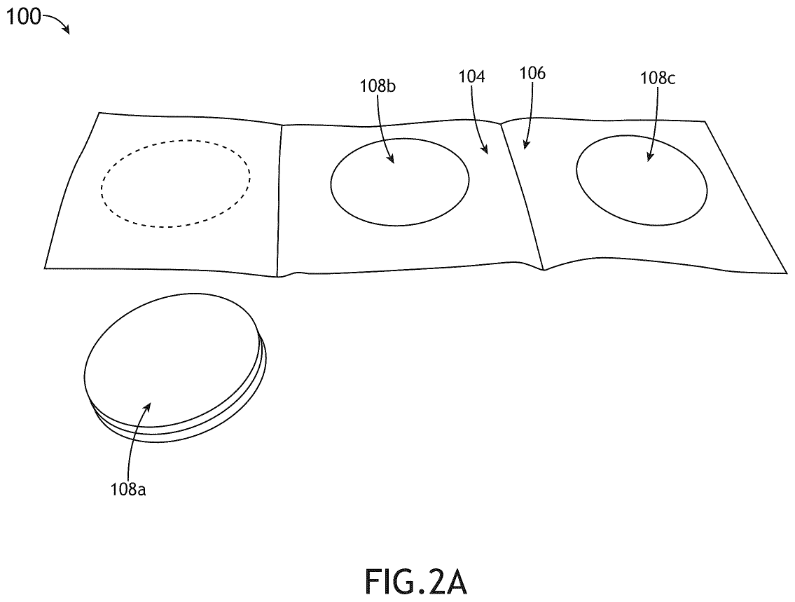

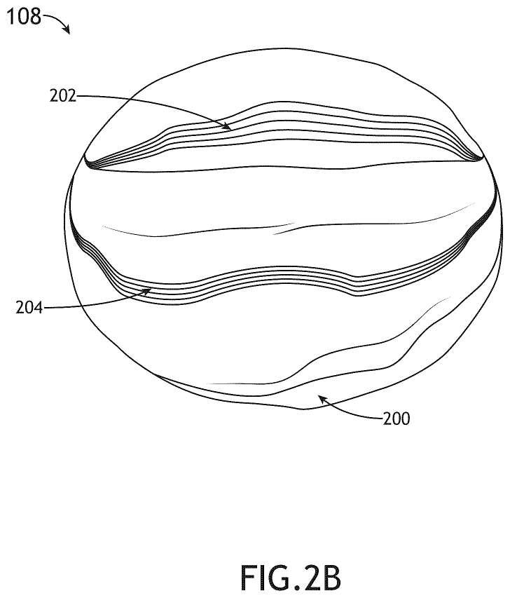

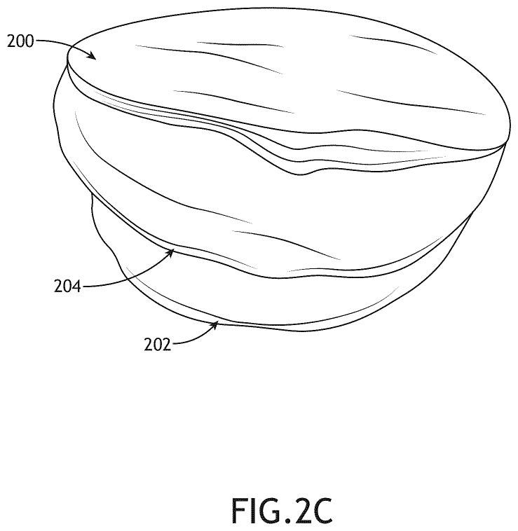

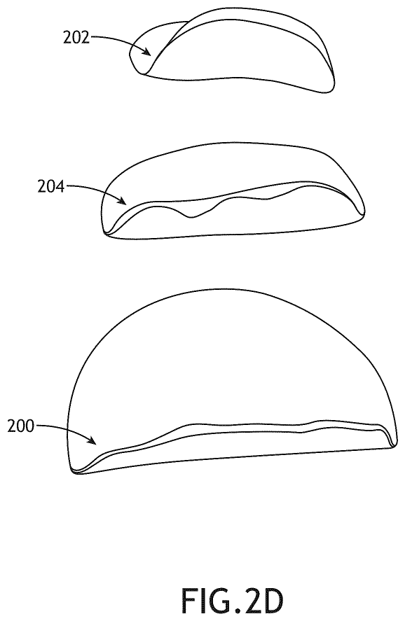

FIG. 2A illustrates the absorbent article 100 with one of the one or more liquid collection units 108 excised and placed adjacent to the absorbent article 100, in accordance with one or more embodiments of the disclosure. The one or more liquid collection units 108 may be free-floating within the absorbent article 100, or may be adhered to the liquid absorbent core 116 and/or the wick material 106. For example, the one or more liquid collection units 108 may be adhered directly to the front side 118 of the liquid absorbent core 116 via an adhesive. In another example, the liquid collection unit may be contained within a region of the absorbent article 100 via a restrictive seam. In embodiments, the one or more liquid collection units 108 are partially or totally wrapped. For example, the one or more collection units 108 may be partially or totally wrapped with one or more layers of permeable nonwoven material (e.g., the nonwoven material used for the wick material 106).

FIGS. 2B and 2C illustrate obverse and reverse perspective views of a partially disassembled (e.g., partially pulled apart) liquid collection unit 108, respectively, in accordance with one or more embodiments of the disclosure. The liquid collection unit 108 is shown as having multiple layers. For example, the liquid collection unit 108 may include a liquid-restrictive layer 200 that is disposed on the front side 118 of the liquid absorbent core 116, one or more initial absorbent materials 202, and a sample absorbent material 204 that collects the second volume of liquid. Because the sample absorbent material 204 and the liquid-restrictive layer 200 may be attached via an adhesive, a sublayer of the sample absorbent material 204 may adhere to the liquid-restrictive layer 200, as shown in FIG. 2B. The sample absorbent material 204 may include multiple layers. For example, the sample absorbent material 204 may include two layers, three layers, or four or more layers.

In embodiments, the initial absorbent material 202 is positioned adjacent to the wick material 106. For example, when the second volume of urine insults the wick material 106, midstream urine will be absorbed by the one or more initial absorbent materials. The portion of the midstream of urine may flow through the initial absorbent material 202 to be absorbed by the sample absorbent material 204 as the second volume. Once the second volume is absorbed by the sample absorbent material 204, the urine is stably stored and prevented from flowing further downward to the liquid absorbent core 116 by the liquid-restrictive layer 200, and the initial absorbent material 202 reduces contamination of the sample absorbent material 204 and prevents the sample absorbent material 204 from drying out, particularly after the absorbent article 100 has been removed from the subject. The second volume remains in the sample absorbent material 204 until the sample absorbent material 204 is removed and processed.

The initial absorbent material 202 and/or the sample absorbent material 204 may include any type of material than absorbs urine and allow excess urine to flow to the sample absorbent material 204. For example, the initial absorbent material 202 may include a fibrous material such as cotton. For instance, the initial absorbent material 202 and/or the sample absorbent material 204 may include an amount of 50% cotton, 90% cotton, more than 90% cotton, or 100% cotton. Other materials in the initial absorbent material 202 may include, but are not limited to, wood pulp, polyester, and rayon, or mixes thereof (e.g., a polyester-rayon mix, a polyester-cotton mix). In embodiments, the initial absorbent material 202 and/or the sample absorbent material 204 includes an absorbent textile including, but not limited to, cotton, rayon, and polyester.

In embodiments, the initial absorbent material 202 and/or the sample absorbent material 204 include a preservative that preserves the collected second volume of urine. The preservative may include, but is not limited to, boric acid, acetic acid, sulfamic acid, sodium hydroxide, oxalic acid, toluene, chelating agents (e.g., EDTA), monalazone disodium, sodium bicarbonate and hydrochloric acid. For example, the initial absorbent material 202 may include and/or be treated with boric acid.

FIG. 2D illustrates a disassembled liquid collection unit 108, in accordance with one or more embodiments of the disclosure. In embodiments, the sample absorbent material 204 is removable from the more liquid collection units 108. For example, after the absorbent article 100 is removed from the subject, the liquid collection unit 108 may be removed from the absorbent article 100 (e.g., by unfolding, tearing or cutting of the wick material 106). The liquid collection unit 108 may then be pulled apart, revealing the sample absorbent material 204. The sample absorbent material 204 may then be isolated from the liquid collection unit and processed.

As described herein, the liquid-restrictive layer 200 prevents and/or inhibits urine collected within the sample absorbent material 204 from being in contact with, and being absorbed by, the liquid absorbent core 116. For example, the liquid-restrictive layer 200 may prevent absorbent polymer, such as SAP, within the liquid absorbent core 116 from absorbing urine from the sample absorbent material 204, even after the absorbent article 100 has been removed from the patient. The liquid-restrictive layer 200 may include any type of impermeable or semi-permeable material including, but not limited to, polyurethane.

In embodiments, after urine insult of the absorbent article by the subject, the urine is extracted from the sample absorbent material 204 for testing. The urine may be extracted from the sample absorbent material 204 by any means. For example, the urine-containing sample absorbent material 204 may be placed or inserted into a barrel of a syringe. A plunger is then forced into the syringe. Compression of the sample absorbent material 204 by the plunger and the end of the syringe barrel releases urine from the sample absorbent material 204, where it flows through the barrel tip. In another example, the urine-containing sample absorbent material 204 may be placed in a centrifuge tube and spun at high speed, releasing the urine from the sample absorbent material 204, which can be removed by a pipette. In another example, the urine-containing sample absorbent material 204 may be placed in a retention column or filter column within a centrifuge tube, then placed in the centrifuge and spun. Here, the material of the sample absorbent material 204 will be retained in the retention column, while the urine will flow through the retention column to the bottom of the centrifuge tube. In another example, the urine is extracted from the sample absorbent material 204 via a solid phase extraction (SPE) method.

In embodiments, the absorbent article 100 is configured to wick and/or distribute a first volume of liquid away from the liquid collection unit 108 via the wick material 106 and collect a second volume within the sample absorbent material 204. For example, the absorbent article 100 may be configured to distribute a first volume of approximately 5 ml and collect approximately 2 ml of a second volume in the sample absorbent material 204. In another example, the absorbent article 100 may be configured to distribute a first volume of approximately 10 ml and collect approximately 2 ml of a second volume in the sample absorbent material 204.

It is to be understood that the second volume refers to a volume of urine that is absorbed by the sample absorbent material 204, which includes mid-stream urine. Once the sample absorbent material 204 receives the second volume, the subject will likely continue to urinate, with excess mid-stream urine being absorbed by the liquid absorbent core 116 or the garment worn by the subject. Therefore, while the second volume of urine includes mid-stream urine, the second volume of urine may not include all mid-stream urine from the subject.

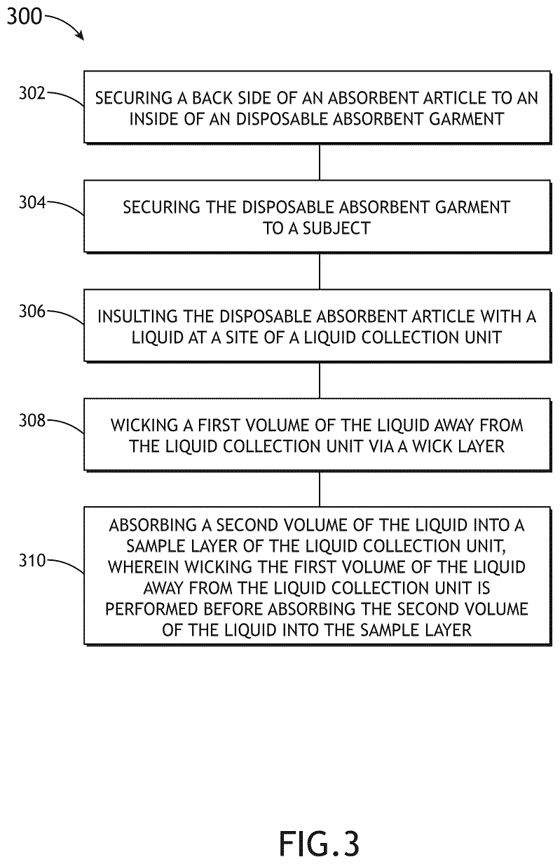

FIG. 3 illustrates a process flow diagram depicting a method 300 for acquiring a urine specimen, in accordance with one or more embodiments of the disclosure. The method 300 generally describes the collection of a urine specimen that has been absorbed by the absorbent article 100.

In embodiments, the method 300 includes a step 302 of securing a back side 118 of the absorbent article 100 to an inside of a disposable absorbent garment. Securing the absorbent article 100 may include positioning the absorbent article 100 between the subject and the disposable absorbent garment, such as a diaper. Securing the absorbent article may also include removing the adhesive strip cover 112 and adhering the absorbent article to the disposable absorbent garment via the adhesive strip.

In embodiments, the method 300 includes a step 304 of insulting the absorbent article 100 with a liquid at a site of a liquid collection unit 108. For example, the subject wearing a disposable absorbent garment with the absorbent article 100 may urinate onto the absorbent article 100 at a position directly above one of the one or more liquid collection units 108, with the urine initially contacting the wick material 106.

In embodiments, the method 300 includes a step 306 of wicking a first volume of the liquid away from the liquid collection unit via a wick material. For example, the wick material may wick away the first volume of urine from the area immediately above the one or more liquid collection units 108 and distribute the urine along the surface of the absorbent article 100. For instance, a first volume of at least five ml of urine may be wicked away from the liquid distribution unit 108 and distributed along the absorbent article 100.

In embodiments, the method 300 includes a step 308 of absorbing a second volume of the liquid into the sample absorbent material 204 of the liquid collection unit 108, wherein wicking the first volume of the liquid away from the liquid collection unit is performed before absorbing the second volume of the liquid into the sample absorbent material 204. Because the first volume of urine may include urine from the urethra that may contain microbes that would confound a UTI test, it is important that the second volume of urine (e.g., containing the midstream urine sample) be collected separately from the first volume of urine (e.g., the first catch sample). Therefore, after the first volume of urine is wicked away from the liquid collection unit 108 and distributed, the second volume of urine is able to enter into the liquid collection unit 108. The urine is first absorbed by the one or more initial absorbent materials 202, then, overflows into the sample absorbent material 204. Once the sample absorbent material is saturated, the entire liquid collection unit 108 is saturated, and the urine will be distributed to other parts of the absorbent article 100 and/or absorbent garment. Once the sample absorbent material 204 has received the second volume of urine, the absorbent article is then removed from the subject. Because the wick material 106 above the liquid collection unit 108, may include a small amount of the first volume of urine when the wick material 106 is insulted with midstream urine, a small amount of the first volume may be carried to the sample absorbent material 204. However, the small amount will not cause the collected second volume to be ineffective when recovered and tested.

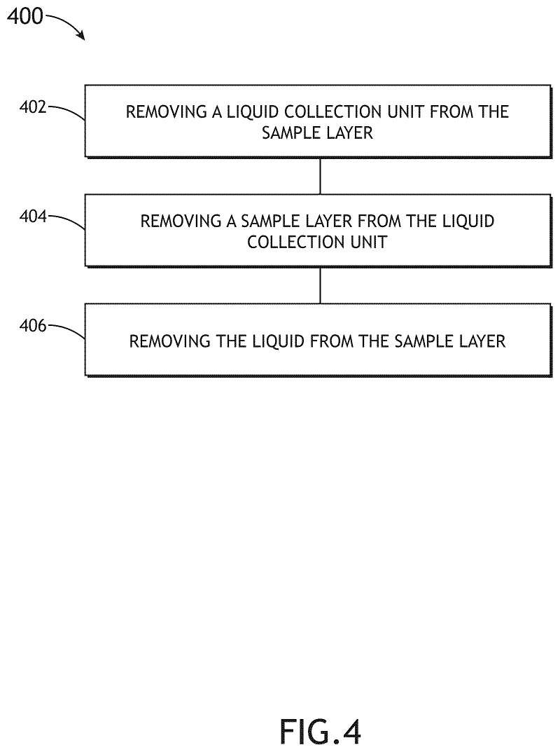

FIG. 4 illustrates a process flow diagram depicting a method 400 for recovering the second volume from the absorbent article 100, in accordance with one or more embodiments of the disclosure. Recovering the second volume may be performed on-site (near the bedside) or at a testing facility. For example, the entire absorbent article 100 may be packaged and sent to the testing facility for processing.

In embodiments, the method 400 includes a step 402 of removing the liquid collection unit 108 from the absorbent article 100. For example, the wick material 106 may be pulled away, torn away, or cut, exposing the liquid collection unit 108, which can then be pulled from the absorbent article 100.

In embodiments, the method 400 includes a step 404 of removing the sample absorbent material from the liquid collection unit 108. For example, the restrictive layer 200 and/or the one or more initial absorbent materials 202 can be peeled back, exposing the sample absorbent material 204. The sample absorbent material 204 may then be pulled from the liquid collection unit 108.

In embodiments, the method 400 includes a 406 step of removing the liquid from the sample absorbent material 204. For example, urine can be removed from the sample absorbent material by placing the sample absorbent material 204 in a syringe barrel and applying a plunger to the syringe barrel, as described above. The urine may also be recovered via centrifugation or other liquid recovery methods.

In embodiments, a method for acquiring a specimen may include steps taken after the absorbent article 100, the liquid collection unit 108, and/or a swab containing the second volume (e.g., mid-stream urine) from the absorbent article has been sent to a testing facility. For example, a method may include one or more steps of: receiving a swab containing a mid-stream urine specimen from a liquid collection unit 108 of an absorbent article 100; placing the swab in a syringe barrel; applying a plunger to the syringe barrel; and collecting the mid-stream urine specimen. The collected mid-stream urine specimen may then be used for testing. The swab may be sent directly to (e.g., and received by) the testing facility. Alternatively, the absorbent article 100 or the liquid collection unit 108 may be sent to the testing facility, and a swab is then used to collect the mid-stream urine specimen, where a technician may receive the swab and proceed to the next step of the method. Collecting the mid-stream urine specimen may include collecting two to five milliliters of mid-stream urine.

As used throughout, “at least one” means one or a plurality of; for example, “at least one” may comprise one, two, three, . . . , one hundred, or more. Similarly, as used throughout, “one or more” means one or a plurality of; for example, “one or more” may comprise one, two, three, . . . , one hundred, or more. Further, as used throughout, “zero or more” means zero, one, or a plurality of; for example, “zero or more” may comprise zero, one, two, three, . . . , one hundred, or more.

It is to be understood that embodiments of the methods disclosed herein may include one or more of the steps described herein. Further, such steps may be carried out in any desired order and two or more of the steps may be carried out simultaneously with one another. Two or more of the steps disclosed herein may be combined in a single step, and in another embodiment, one or more of the steps may be carried out as two or more sub-steps. Further, other steps or sub-steps may be carried in addition to, or as substitutes to one or more of the steps disclosed herein.

In the present disclosure, the methods, operations, and/or functionality disclosed may be implemented as sets of instructions or software readable by a device. Further, it is understood that the specific order or hierarchy of steps in the methods, operations, and/or functionality disclosed are examples of exemplary approaches. Based upon design preferences, it is understood that the specific order or hierarchy of steps in the methods, operations, and/or functionality can be rearranged while remaining within the scope of the inventive concepts disclosed herein. The accompanying claims may present elements of the various steps in a sample order and are not necessarily meant to be limited to the specific order or hierarchy presented.

Before explaining at least one embodiment of the inventive concepts disclosed herein in detail, it is to be understood that the inventive concepts are not limited in their application to the details of construction and the arrangement of the components or steps or methodologies set forth in the following description or illustrated in the drawings. In the following detailed description of embodiments of the instant inventive concepts, numerous specific details are set forth in order to provide a more thorough understanding of the inventive concepts. However, it will be apparent to one of ordinary skill in the art having the benefit of the instant disclosure that the inventive concepts disclosed herein may be practiced without these specific details. In other instances, well-known features may not be described in detail to avoid unnecessarily complicating the instant disclosure. The inventive concepts disclosed herein are capable of other embodiments or of being practiced or carried out in various ways. Also, it is to be understood that the phraseology and terminology employed herein is for the purpose of description and should not be regarded as limiting.

As used herein a letter following a reference numeral is intended to reference an embodiment of the feature or element that may be similar, but not necessarily identical, to a previously described element or feature bearing the same reference numeral (e.g., 1, 1a, 1b). Such shorthand notations are used for purposes of convenience only and should not be construed to limit the inventive concepts disclosed herein in any way unless expressly stated to the contrary.

Further, unless expressly stated to the contrary, “or” refers to an inclusive or and not to an exclusive or. For example, a condition A or B is satisfied by any of the following: A is true (or present), and B is false (or not present), A is false (or not present), and B is true (or present), and both A and B are true (or present).

In addition, use of the “a” or “an” are employed to describe elements and components of embodiments of the instant inventive concepts. This is done merely for convenience and to give a general sense of the inventive concepts, and “a” and “an” are intended to include one or at least one and the singular also includes the plural unless it is obvious that it is meant otherwise.

Finally, as used herein, any reference to “one embodiment,” or “some embodiments” means that a particular element, feature, structure, or characteristic described in connection with the embodiment is included in at least one embodiment of the inventive concepts disclosed herein. The appearances of the phrase “in some embodiments” in various places in the specification are not necessarily all referring to the same embodiment, and embodiments of the inventive concepts disclosed may include one or more of the features expressly described or inherently present herein, or any combination of sub-combination of two or more such features, along with any other features which may not necessarily be expressly described or inherently present in the instant disclosure.

What is claimed:

1. An absorbent article comprising:
a wick material positioned to receive a liquid insult, wherein the liquid insult comprises a first volume and a second volume;
one or more liquid collection units positioned underneath the wick material, wherein the wick material distributes the first volume away from the one or more liquid collection units, the one or more liquid collection units comprising:
an initial absorbent material configured to receive the second volume flowing through the wick material;
a sample absorbent material configured to:
receive the second volume flowing through the initial absorbent material; and
store the second volume; and
a liquid-restrictive layer; and
a liquid absorbent core, wherein the one or more liquid collection units are disposed on or in a front side of the liquid absorbent core, wherein the liquid-restrictive layer of the one or more liquid collection units inhibits liquid collected within the sample absorbent material from contacting the liquid absorbent core.

2. The absorbent article of claim 1, wherein the wick material further covers a back side of the initial absorbent material.

3. The absorbent article of claim 1, further comprising an adhesive layer applied on an exterior side.

4. The absorbent article of claim 1, wherein the liquid absorbent core comprises an absorbent agent.

5. The absorbent article of claim 4, wherein the absorbent agent comprises superabsorbent polymer (SAP).

6. The absorbent article of claim 1, wherein the sample absorbent material comprises an absorbent textile.

7. The absorbent article of claim 6, wherein the absorbent textile comprises at least one of cotton, rayon, and polyester.

8. The absorbent article of claim 7, wherein the absorbent textile comprises more than 90% cotton.

9. The absorbent article of claim 1, wherein the sample absorbent material is configured to be removed from the one or more liquid collection units after the liquid insult and inserted into a syringe barrel.

10. The absorbent article of claim 1, wherein when the sample absorbent material is removed after a saturating liquid insult a recovery volume equal to or more than two milliliters of liquid can be removed from the sample absorbent material.

11. The absorbent article of claim 1, wherein the first volume is equal to or more than five milliliters.

12. The absorbent article of claim 1, wherein when the sample absorbent material is removed after a saturating liquid insult a recovery volume equal to or more than two milliliters of the second volume can be removed from the sample absorbent material, wherein the first volume is equal to or more than five milliliters.

13. The absorbent article of claim 1, wherein the initial absorbent material comprises a preservative.

14. The absorbent article of claim 13, wherein the preservative comprises at least one of boric acid, monalazone disodium, and sodium bicarbonate.

15. The absorbent article of claim 1, wherein the initial absorbent material comprises at least 90% cotton.

16. The absorbent article of claim 1, wherein the liquid-restrictive layer comprises a nonpermeable layer.

17. The absorbent article of claim 1, wherein the liquid-restrictive layer comprises polyurethane.

18. The absorbent article of claim 1, wherein the wick material comprises nonwoven material.

19. The absorbent article of claim 1, wherein the absorbent article is configured as a wearable garment.

20. A method for acquiring a urine specimen comprising:
receiving a swab containing a mid-stream urine specimen from a liquid collection unit of an absorbent article;
placing the swab in a syringe barrel;
applying a plunger to the syringe barrel; and
collecting the mid-stream urine specimen.
